(19) United States
(12) Patent Application Publication
(10) Pub. No.: US 2005/0074424 A1
(43) Pub. Date: Apr. 7, 2005

(54) Using polyion polymers with glucose infusion for a cancer selective chemotherapy compound and method

(76) Inventor: Lev Salnikov, Brookline, MA (US)

Correspondence Address: Donald W. Meeker;Patent Agent, 924 East Ocean Front #E, Newport Beach, CA, 92661, US

(21) Appl. No.: 10/679,039

(22) Filed: Oct. 3, 2003

(51) Int. Cl. A61K 31/785; A61K 31/70

(52) U.S. Cl. 424/78.12; 424/78.27; 514/23

(57) ABSTRACT

A compound and method are provided for combining a chemotherapeutic drug with a polyion polymer for selective chemotherapy delivery. A glucose solution is combined with the polyion polymer, which allows the glucose solution and polyion polymer to be infused into a body containing cancer cells. The glucose solution is capable raising the pH level of cancer cells to a pH of 6.0 to 4.0. The polyion polymer reacts to different pH levels. The acidic environment of the cancer cell causes the polyion polymer to take on an “open line” form, thereby releasing the chemotherapeutic drug to selectively attack the cancer cells. In the neutral or low pH environment of healthy cells the chemotherapeutic drug is retained in an inactive form within the polyion polymer, which remains in the globular closed form, thereby leaving healthy tissue undamaged.

### BACKGROUND OF THE INVENTION

1. Field of the Invention

The present invention relates to compounds and methods for delivery to cancerous cells and in particular to pH-sensitive polyion polymers used with glucose infusion for selective cancer chemotherapy.

2. Description of the Prior Art

Many scientists around the world are looking for a better way to deliver a proven cancer therapy to a tumor without disturbing healthy tissue. It is well known that any tumor tissue uses glucose in large amounts, which produces lactic acid, as a result the intracellular pH levels in tumors lean toward the acidic side. An infusion with additional amount glucose may be used to make a larger difference between the pH readings of normal and tumor tissues. This is a universal condition for all types of cancer cells, therefore there is a possibility to use this peculiarity for selective cancer chemotherapy. Polyion polymers can be used for this purpose.

The controlled release of pharmaceuticals after their administration is under intensive development. Pharmaceuticals have also been complexed with a variety of biologically-labile polymers to delay their release from depots. Biologically active molecules may be assisted by a reversible formation of covalent bonds. Quite often, it is found that the drug administered to a patient is not the active form of the drug, but is what is a called a prodrug that changes into the actual biologically active compound upon interactions with specific enzymes inside the body. In particular, anticancer drugs are quite toxic and are administered as prodrugs, which do not become active until they come in contact with the cancerous cell.

Recent studies have found that pH in solid tumors is lower than in normal tissue. Hence, the use of pH-sensitive polymers for tumor targeting is justified. Liposomes were also used as drug delivery vehicles for low molecular weight drugs and macromolecules such as amphotericin B for systemic fungal infections and candidiasis. Inclusion of anti-cancer drugs such as adriamycin have been developed to increase their delivery to tumors and reduce it to other tissue sites (e.g. heart) thereby decreasing their toxicity. Ph-sensitive polymers have been used in conjunction with liposomes for the triggered release of an encapsulated drug.

U.S. Patent Application No. 20030026841, published Feb. 6, 2003 by Trubetskoy, puts forth compositions and methods for drug delivery using pH sensitive molecules. A polyampholyte is utilized in a condensed polynucleotide complex for purposes of nucleic acid delivery to a cell. The complex can be formed with an appropriate amount of positive and/or negative charge such that the resulting complex can be delivered to the extravascular space and may be further delivered to a cell.

U.S. Patent Application No. 20010024829, published Sep. 27, 2001 by Wolff, concerns polyampholytes for delivering polyions to a cell. A polyampholyte is utilized in a condensed polynucleotide complex for purposes of nucleic acid delivery to a cell. The complex can be formed with an appropriate amount of positive and/or negative charge such that the resulting complex can be delivered to the extravascular space and may be further delivered to a cell.

U.S. Pat. No. 6,338,859, issued Jan. 15, 2002 to Leroux, discloses novel polymeric micelles that are used to deliver therapeutic agents, including anti tumor drugs.

U.S. Pat. No. 6,232,295, issued May, 15, 2001 to Kayyem, indicates a delivery vehicle is described that is capable of being specifically bound to and taken into targeted cells, delivering numerous paramagnetic ions for magnetic resonance imaging (MRI) of the cells. The delivery vehicle comprises a polymeric molecule that has a net positive charge complexed with another polymeric molecule, which has a net negative charge. Cell targeting moieties and MRI contrast agents are attached to one or both of the polymeric molecules. In one embodiment, the polymeric molecule that has a net negative charge is a nucleic acid. Thus, the delivery vehicles can be used in clinical protocols in which nucleic acids for gene therapy and agents for MRI contrast are co-transported to specific cells allowing medical imaging monitoring of nucleic acid delivery.

U.S. Pat. No. 6,126,964, issued Oct. 3, 2000 to Wolff, puts forth a method of forming polymers in the presence of nucleic acid using template polymerization. Also shown is a method of in which the polymerization occurs in heterophase systems. These methods can be used for the delivery of nucleic acids, for condensing the nucleic acid, for forming nucleic acid binding polymers, for forming supramolecular complexes containing nucleic acid and polymer, and for forming an interpolyelectrolyte complex.

U.S. Pat. No. 5,635,487, issued Jun. 3, 1997 to Wolff, concerns amphipathic, micellar delivery systems for biologically active polyions. A composition is described that comprises a population of micelles wherein each micelle comprises at least one amphipathic compound layer that surrounds a non-aqueous core that contains a polyion. Also provided are methods of preparing such a composition and the uses of such compositions for delivering biologically active polyions to cells.

What is needed is a selective cancer chemotherapy delivery system using polyion polymers with glucose infusion so that the chemotherapy drug is only delivered to the cancer cell which transform glucose in large amounts to produce lactic acid which releases the chemotherapy drug from the polyion polymers to attack only the cancer cell.

### SUMMARY OF THE INVENTION

An object of the present invention is to provide a selective cancer chemotherapy delivery system using polyion polymers with glucose infusion so that the chemotherapy drug is only delivered to the cancer cell which transform glucose in large amounts to produce lactic acid which releases the chemotherapy drug from the polyion polymers to attack only the cancer cell.

Another object of the present invention is to provide a selective cancer chemotherapy delivery system that will increase the survival rate of cancer, by attacking only cancerous cells, leaving healthy cells undamaged.

One more object of the present invention is to provide a selective cancer chemotherapy delivery system that will better the cancer patient's quality of life during chemotherapy treatment, with less tissue damage the patient will experience less severe side effects.

In brief, the polyion polymer, for example polydextrose, is formed in a line from hundreds of units. Each unit has different amounts of [+] and [−] radicals. In other words, part of the units has an electrical charge, plus or minus. The correlation between the amount of electrical charge inside this polymer and the pH of surrounding solution makes one of an appropriate spatial form, either “globular” or “open line”.

It is possible to create such a polymer, which will be in “line” form in a solution with acid reaction (pH 6.0-4.0 as in tumor tissue) and in “globular” form in neutral or low alkaline (pH 7.4 as in normal tissue).

Active chemotherapeutic drugs, such as nitros-metyl-urea or others, can be added to the polymer. The polymer will exist in “open line” form in any tissues with an acid pH. In “open line” form the chemotherapeutic agents which are connected to the polymer are allowed to become free and in active form. In a solution with a neutral pH the polymer exists in a “globular” form and keeps the chemotherapeutic drug in an inactive form inside of the polymer molecule. Due to the reaction of the polymer to the different pH levels, selective delivery of chemotherapeutic agents may occur, thereby affecting the tumor tissue while leaving healthy tissue unharmed.

An advantage of the present invention is that active chemotherapeutic drugs may be delivered directly to cancer cells.

Another advantage of the present invention is that the chemotherapeutic drugs will not damage healthy tissue.

An additional advantage of the present invention is in raising the rate of cancer patient survival.

One more advantage of the present invention is in giving a cancer patient a better quality of life while undergoing chemotherapy.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a diagrammatic view of a polyion polymer maintaining a globular form to hold in the chemotherapy drug in a normal cellular environment with a pH 7.0-7.4;

FIG. 2 is a diagrammatic view of the polyion polymer of FIG. 1 transformed into an open line form to release the chemotherapy drug in an acidic cancer cell environment with a pH 6.0-4.0 after a glucose infusion.

### BEST MODE FOR CARRYING OUT THE INVENTION

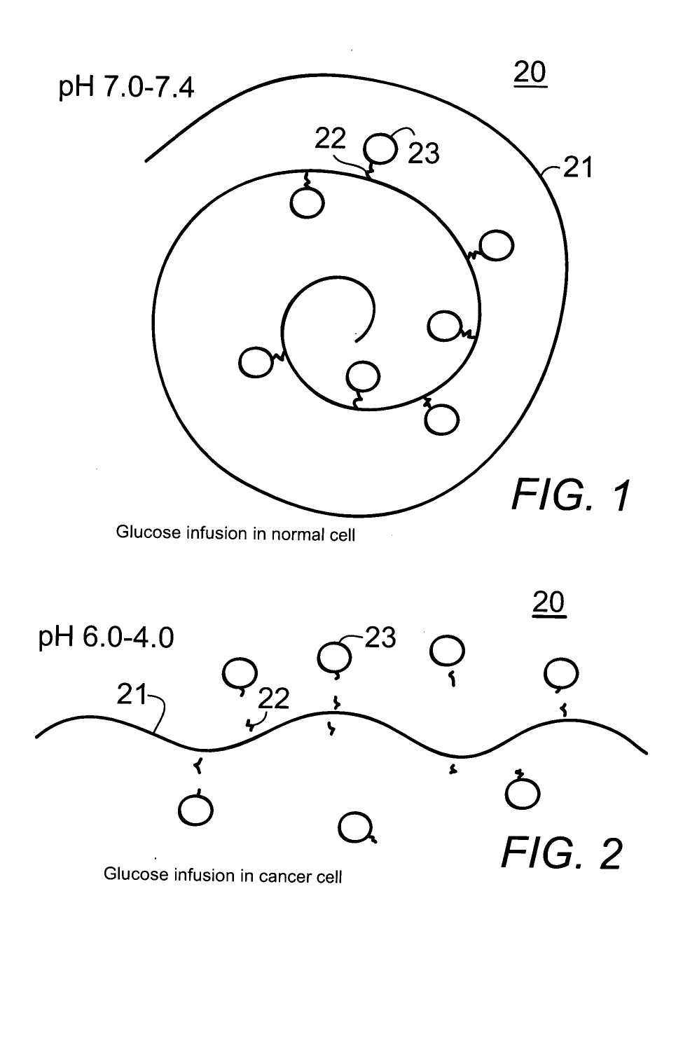

In FIGS. 1 and 2, a compound 20 for cancer cell selective chemotherapy is provided, which comprises a polyion polymer 21 formed in a line from hundreds of units with different amounts of plus and minus radicals. The hundreds of units with different amounts of plus and minus radicals preferably comprise polydextrose. The polyion polymer 21 takes a globular closed form in a neutral and low alkaline solution, as shown in FIG. 1. The polyion polymer 21 takes an open line form in an acid environment, as shown in FIG. 2.

The compound 20 for cancer cell selective chemotherapy also includes a chemotherapeutic drug 23, which may comprise nitros-metyl-urea. The chemotherapeutic drug 23 is combined with the polyion polymer 21 so that the chemotherapeutic drug 23 is retained by chemical bonds 22 in an inactive form within the polyion polymer 21 in the globular closed form, shown in FIG. 1. The chemotherapeutic drug 23 is released in a free active form when the chemical bond 22 is broken from the polyion polymer 21 in the open line form, as shown in FIG. 2.

The compound 20 for cancer cell selective chemotherapy also comprises a glucose solution (not shown) that is combined with the polyion polymer 21. The glucose solution and polyion polymer 21 are capable of being infused into a body containing cancer cells, which produce an acid environment when exposed to glucose. The glucose solution is capable of producing a pH of 6.0 to 4.0 in cancer cells. The polyion polymer 21 maintains a globular closed form in a neutral and low alkaline environment of normal cells retaining the chemotherapeutic drug 23 in an inactive form in the normal cells, as shown in FIG. 1. The polyion polymer 21 transforms into the open line form in a glucose induced acid environment of the cancer cells releasing the chemotherapeutic drug 23 in a free active form in the cancer cells, as shown in FIG. 2, to selectively attack the cancer cells.

In practice, the first step of the method for cancer cell selective chemotherapy comprises forming a polyion polymer 21 in a line from hundreds of units with different amounts of plus and minus radicals. The hundreds of units with different amounts of plus and minus radicals preferably comprise polydextrose. The polyion polymer 21 takes a globular closed form in a neutral and low alkaline solution, as shown in FIG. 1. The polyion polymer 21 takes an open line form in an acid environment, as shown in FIG. 2.

The second step of the method is to combine a chemotherapeutic drug 23, which may comprise nitros-metyl-urea, with the polyion polymer 21. By combining the polyion polymer 21 and the chemotherapeutic drug 23, the chemotherapeutic drug 23 is retained in an inactive form within the polyion polymer 21 in the globular closed form, shown in FIG. 1, or the chemotherapeutic drug 23 is released in a free active form from the polyion polymer 21 in the open line form, shown in FIG. 2.

The third step of the method is to combine a glucose solution (not shown) with the polyion polymer 21 and infuse the glucose solution and polyion polymer 21 into a body containing cancer cells. The cancer cells produce an acid environment when exposed to glucose. The glucose solution is capable of producing an acidic pH of 6.0 to 4.0 in cancer cells. The polyion polymer 21 transforms into an open line form in the glucose induced acid environment of the cancer cells, thereby releasing the chemotherapeutic drug 23 from its chemical bonds 22 in a free active form in the cancer cells, as shown in FIG. 2, to selectively attack the cancer cells. In healthy cell tissue with a neutral or low alkaline pH level the polyion polymer 21 maintains a globular closed form, thereby retaining the chemotherapeutic drug 23 in an inactive form in the normal cells, as shown in FIG. 1, leaving healthy tissue undamaged.

It is understood that the preceding description is given merely by way of illustration and not in limitation of the invention and that various modifications may be made thereto without departing from the spirit of the invention as claimed.

1. A compound for cancer cell selective chemotherapy comprising:
a polyion polymer formed in a line from hundreds of units with different amounts of plus and minus radicals, which polyion polymer takes a globular closed form in a neutral and low alkaline solution and which polyion polymer takes an open line form in an acid environment;
a chemotherapeutic drug combined with the polyion polymer so that the chemotherapeutic drug is retained in an inactive form within the the polyion polymer in the globular closed form and the chemotherapeutic drug is released in a free active form from the polyion polymer in the open line form;
a glucose solution combined with the polyion polymer, the glucose solution and polyion polymer capable of being infused into a body containing cancer cells, which produce an acid environment when exposed to glucose, so that the polyion polymer maintains a globular closed form in a neutral and low alkaline environment of normal cells retaining the chemotherapeutic drug in an inactive form in the normal cells and the polyion polymer transforms into the open line form in a glucose induced acid environment of the cancer cells releasing the chemotherapeutic drug in a free active form in the cancer cells thereby selectively attacking the cancer cells.

2. The compound of claim 1 wherein the glucose solution is capable of producing a pH of 6.0 to 4.0 in cancer cells.

3. The compound of claim 1 wherein the hundreds of units with different amounts of plus and minus radicals comprises polydextrose.

4. The compound of claim 1 wherein the chemotherapeutic drug comprises nitros-metyl-urea.

5. A method for cancer cell selective chemotherapy comprising:
a first step of forming a polyion polymer in a line from hundreds of units with different amounts of plus and minus radicals, which polyion polymer takes a globular closed form in a neutral and low alkaline solution and which polyion polymer takes an open line form in an acid environment;
a second step of combining a chemotherapeutic drug with the polyion polymer so that the chemotherapeutic drug is retained in an inactive form within the polyion polymer in the globular closed form and the chemotherapeutic drug is released in a free active form from the polyion polymer in the open line form;
a third step of combining a glucose solution with the polyion polymer and infusing the glucose solution and polyion polymer into a body containing cancer cells, which produce an acid environment when exposed to glucose, so that the polyion polymer maintains a globular closed form in a neutral and low alkaline environment of normal cells retaining the chemotherapeutic drug in an inactive form in the normal cells and the polyion polymer transforms into the open line form in a glucose induced acid environment of the cancer cells releasing the chemotherapeutic drug in a free active form in the cancer cells thereby selectively attacking the cancer cells.

6. The method of claim 5 wherein the glucose solution is capable of producing a pH of 6.0 to 4.0 in cancer cells.

7. The method of claim 5 wherein the hundreds of units with different amounts of plus and minus radicals comprises polydextrose.

8. The method of claim 5 wherein the chemotherapeutic drug comprises nitros-metyl-urea.
